(19) United States
(12) Patent Application Publication
(10) Pub. No.: US 2011/0138684 A1
(43) Pub. Date: Jun. 16, 2011

(54) Integrated Syngas Fermentation Process and System

(75) Inventor: David R. KRANZ, Katy, TX (US)

(73) Assignee: Chevron U.S.A. Inc., San Ramon, CA (US)

(21) Appl. No.: 12/635,432

(22) Filed: Dec. 10, 2009

(51) Int. Cl. C10L 1/18; C12P 7/06; C12M 1/00

(52) U.S. Cl. 44/451; 435/161; 435/303.1

(57) ABSTRACT

In some embodiments, the present invention relates to methods (processes) of syngas fermentation involving an integral gasification process, and to corresponding systems for carrying out or implementing such methods. In such methods and systems of the present invention, carbon dioxide (CO₂) produced during the fermentation of syngas is directed into the gasifier (e.g., as a motive gas or component thereof) where it enhances carbon monoxide (CO) production and mitigates char production.

### FIELD OF THE INVENTION

This invention relates generally to syngas fermentation, and specifically to a system and method of syngas fermentation involving an integral gasification process and gasifier.

### BACKGROUND

Syngas is a gaseous mixture comprised primarily of hydrogen (H₂) and carbon monoxide (CO), along with some carbon dioxide (CO₂). Syngas has long been used to produce liquid hydrocarbon fuels and other chemicals via Fischer-Tropsch chemistry (see, e.g., M. E. Dry, “The Fischer-Tropsch process: 1950-2000,” Catalysis Today, vol. 71, pp. 227-241, 2002). More recently, however, syngas has found use as a feed for producing ethanol (and other oxygenated organic molecules) via a fermentation process, where high levels of CO are desirable for the production of ethanol. See, e.g., D. Antoni et al., “Biofuels from microbes,” Appl. Microbiol. Biotechnol., vol. 77, pp. 23-35, 2007; R. P. Datar et al., “Fermentation of Biomass-Generated Producer Gas to Ethanol,” Biotechnology and Bioengineering, vol. 86(5), pp. 587-594, 2004; and L. J. Melnichuk et al., United States Patent Application Publication No. 20070270511, published Nov. 22, 2007.

While gasification of biomass to yield syngas has been described previously (see, e.g., G. W. Huber et al., “Synthesis of Transportation Fuels from Biomass: Chemistry, Catalysts, and Engineering,” Chem. Rev., vol. 106, pp. 4044-4098, 2006; and J. Corella et al., “Biomass Gasification with Air in a Fluidized Bed: Exhaustive Tar Elimination with Commercial Steam Reforming Catalysts,” Energy & Fuels, vol. 13, pp. 702-709, 1999), efforts to integrate syngas manufacture and its subsequent fermentation have not previously been described—at least not in a manner that significantly enhances the efficiency and economics of the overall process. Accordingly, any such integration that does enhance the overall efficiency and economics would be of considerable benefit.

### BRIEF DESCRIPTION OF THE INVENTION

The present invention is generally directed to methods (processes) of syngas fermentation involving an integral gasification process, and to corresponding systems for carrying out or implementing such methods. Generally, in such methods and systems of the present invention, carbon dioxide (CO₂) produced during the fermentation of syngas is directed into the gasifier (e.g., as a motive gas or component thereof) where it enhances carbon monoxide (CO) production and mitigates char production.

In some embodiments, the present invention is directed to one or more methods for generating ethanol from biomass, said method(s) comprising the steps of: (a) gasifying biomass to generate a syngas mixture comprising CO, CO₂, and H₂; wherein the gasifying is carried out in a fluidized bed gasifier using a motive and reactive gas stream comprising a mixture of gases; (b) fermenting the syngas mixture to produce ethanol via a fermentation process driven by a population of microorganisms, wherein CO₂ is produced as a by-product of the fermentation; and (c) directing at least a majority portion of the CO₂ produced during the fermenting step into the gasifying step so as to: (i) contribute as a component of the motive and reactive gas in the fluidized bed gasifier; and (ii) enhance the gasifying step, via an equilibrium shift, so as to increase the production of CO and decrease the production of char.

In some or other embodiments, the present invention is directed to one or more systems for generating ethanol from biomass, said system(s) comprising: (a) a source of biomass amenable to gasification; (b) a fluidized bed gasifier in processible communication with said source of biomass and operable for gasifying said biomass, and comprising an integral heating means; (c) a motive and reactive gas mixture supply and stream in processible communication with said fluidized bed gasifier, wherein the motive and reactive gas is operable for reacting with the biomass in the gasifier to yield a syngas mixture; (d) a fermenting chamber comprising a population of microorganisms suitable for effecting the fermentative transformation of syngas to a fermentation product comprising ethanol and CO₂, wherein said fermenting chamber is in processible communication with said gasifier such that it can receive the syngas produced therefrom; and (e) a separator in processible communication with said fermenting chamber, wherein said separator is operable for separating CO₂ from a residual fermentation product balance, and wherein said separator is in processible communication with the motive and reactive gas mixture supply and stream such that at least a majority of the CO₂ produced in the fermenting chamber is incorporated as a component of the motive and reactive gas mixture supply and stream.

In some embodiments, variations on the above-described methods and systems further comprise directing at least a portion of the CO₂ produced in the fermentation sub-process (fermenting chamber) to a photosynthetic biomass growth sub-process (photosynthetic biomass growth chamber) for the production of biomass that can, in turn, be directed back into the method and system at the gasification step (gasifier).

The foregoing has outlined rather broadly the features of the present invention in order that the detailed description of the invention that follows may be better understood. Additional features and advantages of the invention will be described hereinafter which form the subject of the claims of the invention.

### BRIEF DESCRIPTION OF THE DRAWINGS

For a more complete understanding of the present invention, and the advantages thereof, reference is now made to the following descriptions taken in conjunction with the accompanying drawings, in which:

FIG. 1 illustrates, in stepwise fashion, one or more methods of the present invention by which gasification of biomass to syngas is integrated with the fermentation of said syngas; and

FIG. 2 depicts, in flow diagram form, a system that integrates a biomass gasifier with a syngas fermentation chamber and, optionally, a photosynthetic biomass growth chamber, in accordance with some embodiments of the present invention.

### DETAILED DESCRIPTION OF THE INVENTION

### 1. Introduction

Embodiments of the present invention are, at least in some instances, directed to one or more methods (i.e., processes) whereby a syngas fermentation sub-process is integrated with a gasification sub-process. In at least some or other instances, the present invention is additionally or alternatively directed to one or more systems that operably integrate a syngas fermentation sub-processing means with a biomass gasification sub-processing means. Generally, such systems can be seen as comprising the infrastructure needed to carry out and/or implement such methods.

Generally, in such methods and systems of the present invention, carbon dioxide (CO₂) produced during the fermentation of syngas is directed into the gasification sub-process/gasifier (e.g., as a motive gas or component thereof) where it enhances carbon monoxide (CO) production and mitigates char production. While not intending to be bound by theory, it is posited that additional CO₂ shifts the equilibrium of Eq. 1 in such a way as to favor more CO production (i.e., a net gain).

2CO [symbol] CO₂+C(s) (Eq. 1)

In some variational embodiments, such aforementioned methods and systems are further integrated with a photosynthetic biomass growth process (CO₂+hv) and/or chamber for doing same. Biomass grown in such a process/chamber can be introduced into the process/system at the gasification/gasifier stage, representing all or a portion of the biomass being gasified and/or introduced into the gasification chamber.

In some or other such variational embodiments, such aforementioned methods and systems are further integrated with a Fischer-Tropsch (FT) synthetic process, wherein at least a portion of the syngas produced in the gasification sub-process is processed so as to yield high-value hydrocarbon products (e.g., fuels).

### 2. Definitions

Certain terms and phrases are defined throughout this description as they are first used, while certain other terms used in this description are defined below:

The term, “syngas,” as defined herein, refers to a gaseous mixture comprised primarily of CO and H_{z}, along with some CO₂. Syngas is typically produced via the gasification of carbonaceous materials such as coal or biomass (vide infra), wherein the composition of said syngas is at least somewhat dependent on the type of carbonaceous material and the gasification reactants (e.g., steam, air, O₂) so used. Syngas is sometimes referred to as “producer gas,” and the terms will be used interchangeably herein.

“Fischer-Tropsch synthesis,” as defined herein, broadly refers to the synthesis or production of hydrocarbons from syngas by passing a syngas mixture over catalyst at elevated temperatures.

The term “synfuel,” as used herein, refers to fuel products (e.g., gasoline) produced via a Fischer-Tropsch synthetic process.

The term “biomass,” as used herein, refers to biologically-derived carbonaceous material of a renewable nature. Accordingly, fossil fuels are generally excluded from this definition, as they are not “renewable” on a timescale that is amenable to modern processing methods. While it is debatable as to whether “municipal solid waste (MSW)” is either biologically-derived or renewable, for purposes of this discussion, biomass can be broadened to include MSW—to the extent that such material is processibly-integratable with at least some of the method and system embodiments of the present invention.

The term “gasification,” as used herein, generally refers to the process by which carbonaceous material is heated in a suitably-reactive environment so as to yield a syngas mixture.

The term “char,” as used herein, refers to the generally-undesirable carbon (solid) by-product of gasification.

The term “fermentation,” as used herein, refers to microbially-mediated chemical transformation under aerobic or anaerobic conditions, where bacteria and/or fungi are the microorganisms used to provide said transformation. Most syngas fermentation reported in the literature involves bacteria under anaerobic conditions.

The term “photosynthesis,” as defined herein, refers to the biosynthetic conversion of CO₂ and water into biomass using sunlight as an energetic driving force. Plants and algae are sustained (and grow) via this process.

### 3. Methods

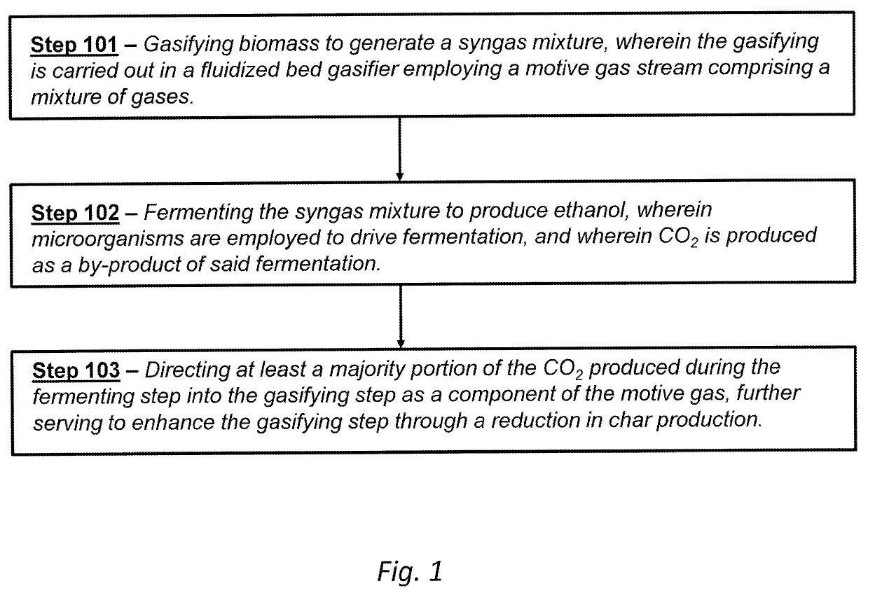

Referring to FIG. 1, in some embodiments, the present invention is directed to one or more methods for generating ethanol from biomass, said method(s) comprising the steps of: (Step 101) gasifying biomass to generate a syngas mixture comprising CO, CO₂, and H₂; wherein the gasifying is carried out in a fluidized bed gasifier using a motive and reactive gas stream comprising a mixture of gases; (Step 102) fermenting the syngas mixture to produce ethanol (CH₃CH₂OH) via a fermentation process driven by a population of microorganisms, wherein CO₂ is produced as a by-product of the fermentation; and (Step 103) directing at least a majority portion of the CO₂ produced during the fermenting step into the gasifying step so as to: (i) contribute as a component of the motive and reactive gas in the fluidized bed gasifier; and (ii) enhance the gasifying step, via an equilibrium shift, so as to increase the production of CO and decrease the production of char.

In some such above-described method embodiments, the biomass is selected from the group consisting of cellulosic biomass, lignocellulosic biomass, lignin, and combinations thereof. Examples of biomass include, but are not limited to, wood, sorgum, rice straw, switchgrass, jatropha, algae, corn, sugarcane, and the like. For additional information on various types of biomass, see G. W. Huber et al., “Synthesis of Transportation Fuels from Biomass: Chemistry, Catalysts, and Engineering,” Chem. Rev., vol. 106, pp. 4044-4098, 2006.

In some or other such embodiments, the biomass is preprocessed prior to it being gasified in the step of gasifying. This can include, but is not limited to, grinding (e.g., to increase surface area), drying, extraction and/or separation, blending of various biomass types, etc. In some embodiments, where lignin represents at least a portion of the biomass being gasified, such lignin can be derived from the waste produced by a paper mill employing a Kraft pulping process (see, e.g., G. Thompson et al., “The treatment of pulp and paper mill effluent: a review,” Bioresource Technology, vol. 77, pp. 275-286, 2001).

Gasification of biomass is well-established and typically takes place in a fluidized bed gasification reactor. Such gasification processes can be catalytic or non-catalytic, suitable examples of which can be found in the literature. See, e.g., J. Gil et al., “Biomass Gasification in Fluidized Bed at Piolt Scale with Steam-Oxygen Mixtures. Product Distribution for Very Different Operating Conditions,” Energy & Fuels, vol. 11(6), pp. 1109-1118, 1997; A. van der Drift et al., “Ten residual biomass fuels for circulating fluidized-bed gasification,” Biomass & Bioenergy, vol. 20, pp. 45-56, 2001. Generally, any gasification process will suffice, such that it suitably provides for a syngas product capable of undergoing fermentation.

In some such above-described method embodiments, a pressure swing adsorption (PSA) O₂ generator is used to supply an O₂ component to the motive and reactive gas stream used in the gasification process. PSA gas separation techniques are known in the art (see, e.g., D. Trommer et al., “Hydrogen production by steam-gasification of petroleum coke using concentrated solar power—I. Thermodynamic and kinetic analyses,” Int. Journal of Hydrogen Energy, vol. 30, pp. 605-618, 2005), and in some such embodiments, use of a PSA O₂ generator can be part of a deliberate effort to minimize N₂ content in the motive and reactive gas stream mixture. While not intending to be bound by theory, it is thought that nitrogen content in the syngas product, possibly in the form of nitrogen oxides (e.g., NOₓ), affect the fermentation of said syngas in an adverse manner. See, e.g., A. Ahmed et al., “Fermentation of Biomass-Generated Synthesis Gas: Effects of Nitric Oxide,” Biotechnology and Bioengineering, vol. 97(5), pp. 1080-1086, 2007. In some or other embodiments, such an O₂ component can be supplied via gas cylinders or cryogenic separation techniques—the selection of which typically being a function of the scale at which the method is implemented.

In some such above-described method embodiments, the step of directing involves a separation sub-process for separating CO₂ from other fermentation products. A variety of such separation techniques exist including, but not limited to, PSA separation, membrane distillation, and cryogenic separation. See, e.g., M. Gryta et al., “Ethanol production in membrane distillation bioreactor,” Catalysis Today, vol. 56, pp. 159-165, 2000; R. Bothast et al., “Biotechnological processes for conversion of corn into ethanol,” Appl. Microbiol. Biotechnol., vol. 67, pp. 19-25, 2005.

The microorganisms (e.g., bacteria and/or fungi) used in the fermentation sub-process are generally limited only in that they should be capable of converting syngas (or components thereof) to ethanol via a fermentative pathway(s). In some such above-described method embodiments, the microorganisms used to drive the fermentation of the fermenting step comprise (as all or part of an overall population of microorganisms) one or more of the following: Clostridium ljungdahlii, Clostridium autoethanogenum, and Clostridium carboxidivorans P7^{T}. Representative microorganisms (microbes) useful in such fermentation sub-processes are further described in the following references: R. Datar et al., “Fermentation of Biomass-Generated Producer Gas to Ethanol,” Biotechnology and Bioengineering, vol. 86(5), pp. 587-594, 2004; A. Henstra et al., “Microbiology of synthesis gas fermentation for biofuel production,” Current Opinion in Biotechnology, vol. 18, pp. 200-206, 2007.

In some embodiments, the fermentation sub-process is additionally tailored by external (to the heretofore described system) perturbations to the environment in which said fermentation takes place. Such perturbations can include, but are not limited to, modifications of the chemical environment, addition or removal of thermal energy, addition of radiative energy, and the like.

In some embodiments, at least a portion of the ethanol produced in the fermentation sub-process is blended with one or more transportation fuels to yield a blended fuel (e.g., State- or Federally-mandated addition of ethanol to gasoline, E10, E25, or E85). See, e.g., Gibbs, United States Patent Application Publication No. 20070256354, published Nov. 8, 2007.

### 4. Systems

Generally, system embodiments of the present invention are used to implement one or more of the method embodiments described in the preceding section. Accordingly, much of the discussion that follows may bear corresponding similarities to the discussion that precedes it. Furthermore, many system configurations not explicitly described can, in fact, be inferred from the description of the method embodiments of Section 3 (above).

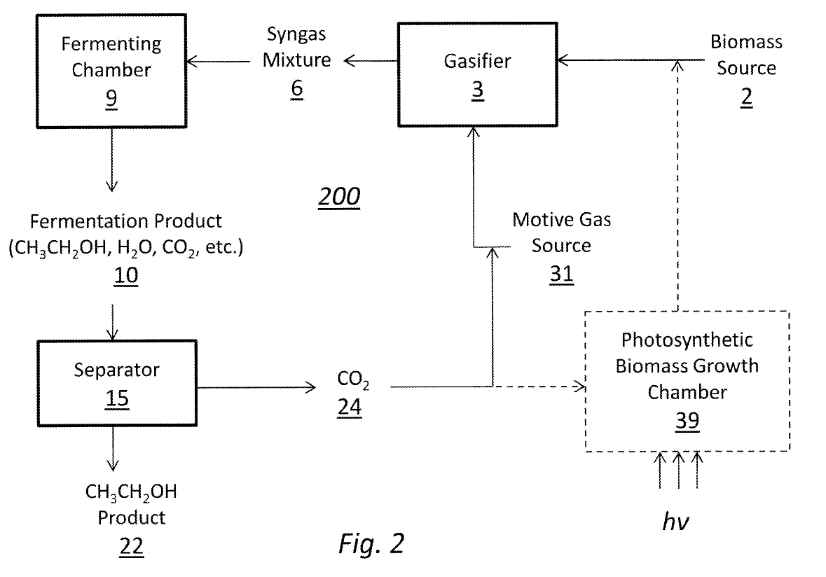

Referring to FIG. 2, in some embodiments the present invention is directed to one or more systems for generating ethanol from biomass, wherein such a system 200 can been seen to comprise a source of biomass 2 amenable to gasification; a fluidized bed gasifier 3 in processible communication with said source of biomass 2 and operable for gasifying said biomass, and comprising an integral heating means (not shown); a motive and reactive gas mixture supply and stream 31 in processible communication with said fluidized bed gasifier 3, wherein the motive and reactive gas is operable for reacting with the biomass in the gasifier to yield a syngas mixture 6; a fermenting chamber 9 comprising a population of microorganisms suitable for effecting the fermentative transformation of syngas to a fermentation product 10 comprising ethanol and CO₂, wherein said fermenting chamber 9 is in processible communication with said gasifier 3 such that it can receive the syngas 6 produced therefrom; and a separator 15 in processible communication with said fermenting chamber, wherein said separator is operable for separating CO₂ from a residual fermentation product balance (10 without CO₂), and wherein said separator 15 is in processible communication with the motive and reactive gas mixture supply and stream 31 such that at least a majority of the CO₂ produced in the fermenting chamber 9 is incorporated as a component of the motive and reactive gas mixture supply and stream 31. Separator 15 can be further operable for isolating an ethanol product 22.

In some such above-described system embodiments, such systems further comprise a biomass pre-processing unit operable for pre-processing at least some of the biomass being fed into the gasifier, and further operable for rendering the biomass more amenable to gasification.

In some such above-described system embodiments, such systems further comprise a pressure swing adsorption (PSA) generator, wherein said PSA generator is in processible communication with the motive and reactive gas mixture supply and stream, and wherein it is operable for supplying an O₂ component of the motive and reactive gas mixture supply and stream. In some or other embodiments, such an O₂ component can be supplied via gas cylinders and/or via cryogenic separation sub-systems (see, e.g., Hansel et al., U.S. Pat. No. 5,076,823; issued Dec. 31, 1991).

In some such above-described system embodiments, said system is engineered and correspondingly operated, so as to minimize the N₂ content of the motive and reactive gas mixture supply and stream. In some such embodiments, the integration of a PSA generator for generating O₂ can provide a means for minimizing such N₂ content.

In some such above-described system embodiments, such systems further comprise a means or subsystem for blending the produced ethanol with one or more transportation fuels, in corresponding agreement with one or more of the methods for doing so described above.

### 5. Variations

In some variations of the above-described method embodiments, such methods can further comprise a step of channeling, or otherwise directing, a portion of the CO₂ produced during the fermenting step to a photosynthetic sub-process for generating biomass. In some such variational method embodiments, the biomass generated by the photosynthetic sub-process is directed into the gasifying step. In some or other such variational method embodiments, a portion of the CO₂ produced during the fermenting step is directed to a photosynthetic sub-process for generating algal biomass. Algae produced in such variational method embodiments can be further processed, in whole or in part, to extract lipids (e.g., for subsequent processing to biodiesel) and/or cellulose (e.g., for subsequent fermentative transformation to ethanol).

In some or other such above-described variational method embodiments, such methods may further comprise a step of processing a portion of the syngas (produced in the gasification sub-process) via Fischer-Tropsch (FT) synthetic procedures (see, e.g., M. E. Dry, “The Fischer-Tropsch process: 1950-2000,” Catalysis Today, vol. 71, pp. 227-241, 2002), so as to yield hydrocarbon products (e.g., synfuels). When the products of such FT sub-processes are (or comprise) synfuel, such synfuel can be blended, in whole or in part, with the ethanol produced by the fermentation sub-process. Such FT-derived synfuel can account for all or part of the fuel with which the produced ethanol is blended.

Referring again to FIG. 2, in some variations of the above-described system embodiments, such systems further comprise a photosynthetic biomass growth chamber 39, wherein said photosynthetic biomass growth chamber 39 is in processible communication (shown by dotted lines) with the separator 15 such that it is functionally operable for receiving a portion of the CO₂ (i.e., a portion of 24) produced in the fermenting chamber 9, and wherein it utilizes this CO₂, together with radiant energy (hv), to grow biomass. In some such variational system embodiments, said photosynthetic biomass growth chamber 39 is placed in processible communication (shown with dotted lines) with the gasifier 3, such that at least a portion of the biomass grown in the photosynthetic biomass growth chamber 39 can be directed into the gasifier 3.

In corresponding agreement with at least some of the above-described variational method embodiments, in some or other such variational system embodiments, there exists, as part of the overall system(s), a FT synthesis sub-system for generating hydrocarbon fuels and/or other useful hydrocarbon chemicals. To the extent that such a sub-system is operable for generating synfuels, there may exist further infrastructure operable for blending such synfuel with the ethanol produced by the fermentation subprocess, so as to produce a blended fuel composition comprising ethanol (vide supra).

In some or other such above-described variational method and/or system embodiments, the gasifier can be other than a fluidized bed gasifier. In such embodiments, the CO₂ produced during fermentation may, or may not, be utilized in, or as, a motive gas.

### 6. Summary

The foregoing describes methods and systems for integrating syngas fermentation with gasification. In such methods and systems of the present invention, CO₂ produced during the fermentation of syngas is directed into the gasifier (e.g., as a motive gas or component thereof) where it enhances CO production and mitigates char production. This in turn leads to a net increase in CO production that equates to greater efficiency in ethanol production. Furthermore, such methods and systems can be further integrated with photosynthetic biomass production and/or Fischer-Tropsch synthesis, so as to provide a high level of flexibility, adaptability, and self-sufficiency.

All patents and publications referenced herein are hereby incorporated by reference to the extent not inconsistent herewith. It will be understood that certain of the above-described structures, functions, and operations of the above-described embodiments are not necessary to practice the present invention and are included in the description simply for completeness of an exemplary embodiment or embodiments. In addition, it will be understood that specific structures, functions, and operations set forth in the above-described referenced patents and publications can be practiced in conjunction with the present invention, but they are not essential to its practice. It is therefore to be understood that the invention may be practiced otherwise than as specifically described without actually departing from the spirit and scope of the present invention as defined by the appended claims.

What is claimed:

1. A method for generating ethanol from biomass, said method comprising the steps of:
a) gasifying biomass to generate a syngas mixture comprising CO, CO₂, and H₂; wherein the gasifying is carried out in a fluidized bed gasifier using a motive and reactive gas stream comprising a mixture of gases;
b) fermenting at least a majority of the syngas mixture to produce ethanol via a fermentation process driven by a population of microorganisms, wherein CO₂ is produced as a by-product of the fermentation; and
c) directing at least a majority portion of the CO₂ produced during the fermenting step into the gasifying step so as to:
i) contribute as a component of the motive and reactive gas in the fluidized bed gasifier; and
ii) enhance the gasifying step, via an equilibrium shift, so as to increase the production of CO and decrease the production of char.

2. The method of claim 1, wherein the step of directing involves a separation sub-process for separating CO₂ from other fermentation products.

3. The method of claim 2, wherein a pressure swing adsorption O₂ generator is used to supply an O₂ component to the motive and reactive gas stream.

4. The method of claim 3 further comprising a step of channeling a portion of the CO₂ produced during the fermenting step to a photosynthetic sub-process for generating biomass.

5. The method of claim 4, wherein the biomass generated by the photosynthetic sub-process is directed into the gasifying step.

6. The method of claim 1, wherein the biomass is preprocessed prior to it being gasified in the step of gasifying.

7. The method of claim 1, wherein the biomass is selected from the group consisting of wood, sorgum, rice straw, switchgrass, jatropha, algae, corn, sugarcane, and combinations thereof.

8. The method of claim 1, wherein a deliberate effort is made to minimize N₂ content in the motive and reactive gas stream mixture.

9. The method of claim 1, wherein the microorganism population used to drive the fermentation of the fermenting step comprises microorganisms selected from the group consisting of Clostridium ljungdahlii, Clostridium autoethanogenum, and Clostridium carboxidivorans P7^{T}.

10. The method of claim 1 further comprising a step of blending the produced ethanol with fuel to yield a blended fuel.

11. The method of claim 1 further comprising a step of synthesizing hydrocarbons via Fischer-Tropsch synthesis with a portion of the syngas produced in the step of gasifying.

12. The method of claim 11, wherein the hydrocarbons synthesized via the Fischer-Tropsch synthesis comprise Fischer-Tropsch product species operable for use as synfuels, and wherein such species are blended with the produced ethanol to yield a blended synfuel.

13. A system for generating ethanol from biomass, said system comprising:
a) a source of biomass amenable to gasification;
b) a fluidized bed gasifier in processible communication with said source of biomass and operable for gasifying said biomass, and comprising an integral heating means;
c) a motive and reactive gas mixture supply and stream in processible communication with said fluidized bed gasifier, wherein the motive and reactive gas is operable for reacting with the biomass in the gasifier to yield a syngas mixture;
d) a fermenting chamber comprising a population of microorganisms suitable for effecting the fermentative transformation of syngas to a fermentation product comprising ethanol and CO₂, wherein said fermenting chamber is in processible communication with said gasifier such that it can receive the syngas produced therefrom; and
e) a separator in processible communication with said fermenting chamber, wherein said separator is operable for separating CO₂ from a residual fermentation product balance, and wherein said separator is in processible communication with the motive and reactive gas mixture supply and stream such that at least a majority of the CO₂ produced in the fermenting chamber is incorporated as a component of the motive and reactive gas mixture supply and stream.

14. The system of claim 13 further comprising a photosynthetic biomass growth chamber, wherein said photosynthetic biomass growth chamber is in processible communication with the separator such that it is functionally operable for receiving a portion of the CO₂ produced in the fermenting chamber, and wherein it utilizes this CO₂, together with radiant energy, to grow biomass.

15. The system of claim 14, wherein said photosynthetic biomass growth chamber is placed in processible communication with the gasifier, such that at least a portion of the biomass grown in the photosynthetic biomass growth chamber can be directed into the gasifier.

16. The system of claim 15 further comprising a pressure swing adsorption generator, wherein said pressure swing adsorption generator is in processible communication with the motive and reactive gas mixture supply and stream, and wherein it is operable for supplying an O₂ component of the motive and reactive gas mixture supply and stream.

17. The system of claim 16 further comprising a biomass pre-processing unit operable for pre-processing at least some of the biomass being fed into the gasifier, and further operable for rendering the biomass more amenable to gasification.

18. The system of claim 17, wherein said system is engineered and corresponding operated so as to minimize the N₂ content of the motive and reactive gas mixture supply and stream.

19. The system of claim 13 further comprising a blending unit, said blending unit being in processible communication with the fermenting chamber and operable for blending at least a portion of the alcohol produced in said chamber with a fuel to yield a blended fuel.

20. The system of claim 13 further comprising a Fischer-Tropsch synthesis chamber in processible communication with said fluidized bed gasifier, such that a portion of the syngas produced in said gasifier can be directed to the Fischer-Tropsch synthesis chamber where it can be processed into synfuel.
